(19) United States
(12) Patent Application Publication
(10) Pub. No.: US 2023/0376792 A1
(43) Pub. Date: Nov. 23, 2023

(54) METHOD AND SYSTEM FOR FACILITATING CLASSIFYING A SEQUENCE

(71) Applicant: Armand Prieditis, McKinleyville, CA (US)

(72) Inventor: Armand Prieditis, McKinleyville, CA (US)

(21) Appl. No.: 17/747,426

(22) Filed: May 18, 2022

(51) Int. Cl. G06N 5/02

(52) U.S. Cl. CPC G06N 5/022

(57) ABSTRACT

One embodiment of the subject matter can facilitate classifying a sequence based on dynamic programming and a probabilistic model that considers both neighbor states and values. This embodiment has several advantages. First, the probabilistic model can be learned from training data. Second, it is more accurate than previous models. Third, it is more efficient that previous methods for both prediction and learning. Embodiments of the subject matter can also be parallelized over the training data to yield a learning time that is linear in the maximum number of elements in the sequences in the training data. Fourth, it is optimal in that it guarantees a prediction that is a most likely one based on the probabilistic model. This guarantee is based on the principle of optimality in dynamic programming and basic probability. Fifth, it leverages locality to improve accuracy rather than throwing away or aggregating information as in feature-based methods.

### BACKGROUND

### Field

The subject matter relates to sequence classification. A sequence is an ordered list of elements. Classification involves determining a class for a sequence.

### Related Art

Sequence classification has many applications, including genomics (classifying genetic sequences), bioinformatics (classifying protein sequences), health informatics (classifying patient data such as electro-cardiograms), information retrieval (to categorize text and documents), speech understanding, and time-series classification.

Attention to sequence classification has grown with the availability of a large amount of publicly available DNA and protein sequences such as from GenBank, the EMBL Nucleotide Sequence Database and the Entrez protein database. This data has been useful to understand the functions of different genes and proteins.

Time series data is another important type of sequence data. The advent of publicly available data sets such as the Time Series Data Library, which includes time series data across 22 domains, such as agriculture, chemistry, health, finance, and industry, has facilitated growth of research in time-series classification.

One approach to sequence classification is feature-based: transform a sequence into a vector of features. After obtaining a vector of features, standard machine learning classification methods such as decision-trees, support vector machines (SVMs), rule-based classifiers and neural networks can be applied to classify a sequence. For example, feature based methods such as k-grams (k-length frequently occur sequences) are widely used for genomic sequence classification. More recent feature-based methods such as Deep Learning automatically extract features. The problem with feature-based methods is that spatial proximity in the original sequence is destroyed and the feature extraction methods do not pay attention to the particular class while extracting features.

Another approach to sequence classification is distance-based: define a distance function to measure the similarity between a pair of sequences. For example, a distance function might return the minimum number of insertions, deletions, and substitutions to transform one sequence to another. Given a distance function, an existing non-parametric classification method such as k-nearest neighbors can be used to find the classes of the closest (distance-wise) neighbors in a database of sequences and their corresponding classes. For example, the predicted class of a sequence can be the most frequent class among the k-nearest neighbors in a database of sequences and their corresponding classes, weighted in proportion to distance. The problem with this approach is that it doesn't generalize to elements that are multivariate (i.e., more than one continuous value at each position in the sequence).

Another sequence classification method is generative model-based, which assumes that sequences in a class are generated by an underlying model of a probability distribution of the sequences in that class. During training, this method learns the parameters of the model. During classification, a test sequence is assigned to the class with the highest likelihood given the test sequence.

A generative model's parameters are typically estimated from a set of training examples. For example, the probability of a particular value at a location in the sequence can be estimated from the observed frequency of that value in the training examples. Typically, a generative model is based on one or more simplifying assumptions. For example, the Naïve Bayes classifier assumes that every element in sequence is conditionally independent given the class. Unfortunately, this conditional independence assumption is almost never true in practice, which results in low accuracy.

In contrast, Markov Models (MMs) and Hidden Markov Models (HMMs) can model local dependence among elements in sequences. For example, a k^{th} order MM can capture the dependence of k elements appearing previously in the sequence. Although MMs have been shown to outperform SVMs with k-grams as input features, an MM can't capture non-linearities in the data. This is because its parameters are averaged over all training examples.

An HMI avoids such averaging by assuming that the data also includes unobserved states, which are labels chosen from a finite set. This can capture non-linearity and thus improve classification performance. HMMs have been applied to gene finding, radiation hybrid mapping, genetic linkage mapping, phylogenetic analysis, and protein secondary structure prediction.

A special type of HMM, called a profile HMI, can be used to classify aligned sequences with three types of states: inserting, matching and deleting states. Profile HMMs suffer from several shortcomings: state transitions follow a strictly left-to-right sequence, training examples must be aligned (which is itself a hard problem to solve), areas of potential matches must be apriori identified (this fixes the model length), and it can converge to suboptimal solutions. More generally, HMMs (not just profile HMMs) assume that a state is dependent on the predecessor state but not the actual values at the predecessor.

Many of the approaches to the classification of time series data are similar to those of genome and protein classification: feature-based, distance-based, and model-based methods. However, time-series classification typically involves only continuous data, sometimes multivariate, and the detection of trends and seasonality (i.e., cycles).

Hence, what is needed is a method and a system for classifying a sequence that doesn't require complex alignment schemes, doesn't require trend and seasonality detection, is multivariate, and can take into account predecessor values, not just predecessor states, for improved accuracy.

### SUMMARY

One embodiment of the subject matter can facilitate classifying a sequence based on dynamic programming and a probabilistic model that considers both neighbor states and values. This embodiment has several advantages. First, the probabilistic model can be learned from training data. Second, it is more accurate than previous models. Third, it is more efficient that previous methods for both prediction and learning. Embodiments of the subject matter can also be parallelized over the training data to yield a learning time that is linear in the maximum number of elements in the sequences in the training data. Fourth, it is optimal in that it guarantees a prediction that is a most likely one based on the probabilistic model. This guarantee is based on the principle of optimality in dynamic programming and basic probability. Fifth, it leverages locality to improve accuracy rather than throwing away or aggregating information as in feature-based methods.

The details of one or more embodiments of the subject matter are set forth in the accompanying drawings and the description below. Other features, aspects, and advantages of the subject matter will become apparent from the description, the drawings, and the claims.

### BRIEF DESCRIPTION OF THE FIGURES

FIG. 1 presents an example system for facilitating sequence classification.

In the figures, like reference numerals refer to the same FIGURE elements.

### DETAILED DESCRIPTION

In embodiments of the subject matter, a sequence comprises one or more elements, each of which can comprise one or more continuous values. A discrete-valued element can be represented as a one-hot vector of continuous values.

In embodiments of the subject matter, the classification task is to predict a class based on a sequence. During operation, embodiments of the subject matter can execute the following procedure.

$$\# for each class c ∈ C determine most likely states$$

$$\# for each element in the sequence$$

$$c ∈ C :$$

$$s ∈ S :$$

$${t}_{1 , s , c} \to {t}_{1 , s , c} ← l ( [ \begin{matrix}{x}_{1} \\ o ( s )\end{matrix} ] \vert o ( c ) , γ : τ , θ : θ , μ , Σ )$$

$${g}_{1 , s , c} ← s$$

$$2 \leq i \leq m :$$

$$s ∈ S :$$

$${t}_{i , s , c} ← {max}_{{s}^{′} ∈ S} \{ l [ \begin{matrix}{x}_{1} \\ o ( s )\end{matrix} ] \vert [ \begin{matrix}{x}_{i - 1} \\ o ( {s}^{′} ) \\ o ( c )\end{matrix} ] , γ : τ , {γ}^{′} : θ , \dot{μ} , \dot{Σ} ) + {t}_{i - 1 , {s}^{′} , c} \}$$

First, embodiments of the subject matter determine the most likely states for each element in the sequence for each class c in a non-empty set of classes C. Here, S corresponds to a non-empty set of states. Typically, the set of states S={1 . . . k}, where k is a positive integer. States are like mixture components in a mixture model: they are merely identifiers that operate like a subclass in a model. More generally, the set of states S can be any finite set of k elements such as {a,b,c,d}. Though the states have different labels, the number of states is the same and hence these two different state sets can be treated equivalently by embodiments of the subject matter. For convenience of implementation, a preferred embodiment of the subject matter comprises states S={1 . . . k}, which is equivalent to any k element set of labels in embodiments of the subject matter.

The expression c∈C: corresponds to a “for” loop that is executed for every class c∈C. Similarly, the expression s∈S: corresponds to a “for” loop that is executed for every state s∈S. For each state, for each element in the sequence, t_{i,s,c} stores the sum of the log maximum likelihood over all elements less than i at state s for class c. Previously computed values of t_{i,s,c} can be used to determine t for larger values of i and other states for the same class c by using dynamic programming, which will be described shortly.

The function l(x|y, a, b, μ,Σ)= [symbol] (x,μₐ+Σ_{a,b}Σ_{b,b}⁻¹(y−μ_{b}), Σ_{a,b}Σ_{b,b}⁻¹Σ_{b,a}), where [symbol] (x,μ,Σ)=ln |Σ|+(x−μ)^{T}Σ⁻¹(x−μ). The function l returns the log of the probability of a conditional multivariate Gaussian distribution. The function [symbol] returns the ln (natural log) of the probability of x in a multivariate Gaussian distribution with mean μ and covariance matrix Σ. (The constants such as π and ½ in [symbol] are removed here because they don't affect the outcome in embodiments of the subject matter). Also ln|Σ| is the natural log of the determinant of Σ, M^{T} is the transpose of matrix M, and Σ⁻¹ is the inverse of a square matrix Σ. The subscripts in the mean μ vector and covariance matrix Σ refer to blocks in these matrices, which are conformably partitioned in a way that will be described shortly.

The assignment

$${t}_{1 , s , c} ← l ( [ \begin{matrix}{x}_{1} \\ o ( s )\end{matrix} ] \vert o ( c ) , γ : τ , θ : θ , μ , Σ )$$

sets the first value of t for state s and class c, where the value x₁ corresponds to first element in input sequence x, for which the class is to be determined. The function o(y) returns the one-hot representation of y. Each class and state can be represented as a one-hot vector. For example, if there are three states, the one-hot vector for the first state can be represented as length 3 column vector with a one in the first position and zeroes elsewhere:

$$[ \begin{matrix}1 \\ 0 \\ 0\end{matrix} ] .$$

A one-hot representation is frequently used in machine learning to handle categorical data. In this representation a k-category variable is converted to a k-length vector, where a 1 in location i of the k-length vector corresponds to the i^{th} categorical variable; the rest of the vector values are 0. For example, if the categories are A, B, and C, then a one-hot representation corresponds to a length three vector where A can be represented as

$$[ \begin{matrix}1 \\ 0 \\ 0\end{matrix} ] , B as [ \begin{matrix}0 \\ 1 \\ 0\end{matrix} ] ,$$

### and C as

$$[ \begin{matrix}0 \\ 0 \\ 1\end{matrix} ]$$

Other permutations of the vector can be used to equivalently represent the same three categorical variables.

Embodiments of the subject matter can leverage both dynamic programming and multivariate Gaussian distributions. It can leverage dynamic programming by using the state, class, and sequence location as an index to save precomputed results. It can leverage multivariate Gaussian distributions by using a one-hot version of the state. For example, t_{1,s,c,} can be precomputed and stored for reuse through dynamic programming, and o(s), the one-hot version of s, and o(c), the one-hot version of c, can be used in a Gaussian distribution because each one-hot version comprises a vector of continuous values (though it is represented as a vector of continuous values, one of which is always a 1 and the rest zeros).

The undotted vectors and matrices correspond to the edge cases for training: the are based on data at the first position in the sequence. The dotted vectors and matrices correspond to the non-edge cases: they are based on data at all subsequence positions in the sequence.

The mean vector μ is conformably partitioned as

$$[ \begin{matrix}{μ}_{γ} \\ {μ}_{τ} \\ {μ}_{θ}\end{matrix} ] ,$$

where μ_{γ} corresponds to the mean of the first element, μ_{τ} corresponds to the mean of the one-hot representation of the state for the first element, and μ_{θ} corresponds to the mean of the one-hot representation of the class for the sequence. The covariance matrix Σ is similarly conformably partitioned as

$$[ \begin{matrix}{Σ}_{γ , γ} & {Σ}_{γ , τ} & {Σ}_{γ , θ} \\ {Σ}_{τ , γ} & {Σ}_{τ , τ} & {Σ}_{τ , θ} \\ {Σ}_{θ , γ} & {Σ}_{θ , τ} & {Σ}_{θ , θ}\end{matrix} ] .$$

Similarly, the second mean vector \dot{μ} is conformably partitioned as

$$[ \begin{matrix}{\dot{μ}}_{γ} \\ {\dot{μ}}_{τ} \\ {\dot{μ}}_{{γ}^{′}} \\ {\dot{μ}}_{{τ}^{′}} \\ {\dot{μ}}_{θ}\end{matrix} ] ,$$

where \dot{μ}_{γ} corresponds to the mean of the i^{th} element (where i>1), \dot{μ}_{τ} corresponds to the mean of the one-hot representation of the state for the i^{th} element, \dot{μ}_{γ′} corresponds to the mean of the i−1^{st} element (The prime (′) notation refers to an immediate predecessor in the sequence), \dot{μ}_{τ′} corresponds to the mean of the one-hot representation of the state for the i−1^{st} element, and \dot{μ}_{θ} corresponds to the mean of the one-hot representation of the class for a sequence.

Also similarly, the second covariance matrix \dot{Σ} is conformably partitioned as

$$[ \begin{matrix}{\dot{Σ}}_{γ , γ} & … & {\dot{Σ}}_{γ , θ} \\ ⋮ & ⋱ & ⋮ \\ {\dot{Σ}}_{θ , γ} & … & {\dot{Σ}}_{θ , θ}\end{matrix} ] .$$

The range notation a:b follows the order of variables that appear in μ, Σ, \dot{μ} and \dot{Σ}. For example, γ′:θ specifies a range of blocks from γ′ to θ, inclusive: γ′, τ′, θ. This range notation is merely a compact and succinct way to specify successive blocks of a conformably partitioned vector or matrix.

The base value of t can be used to set values of t later in the sequence through dynamic programming. An alternative to the base values and μ and Σ is to include a dummy border (a dummy first position that occurs prior to the actual first position in the sequence) and only use \dot{μ} and \dot{Σ}, and the subsequent “for” loop, which will be described shortly.

Although such dummy borders are common in image processing to reduce code, the problem with dummy borders is that a dummy state is required for those edges as well as dummy values at the location associated with the dummy. Zeros are often used as for such values associated with dummy borders, but this can bias the values of \dot{μ} and \dot{Σ}, especially if zeros are actual values in the rest of the sequence.

A disadvantage of using edge cases (i.e., not using dummies) is that for learning, statistically, there are less edge cases in training data. For example, with n k-length sequences, there will only be n edge cases but n×k interior cases. However, in the spirit of greater clarity and potentially improved accuracy, description of embodiments of the subject matter here avoid minor tricks such as a dummy border to reduce the amount of code.

The expression 2≤i≤m: corresponds to a “for” loop that loops through values of i from 2 to m, inclusive, where m is the number of elements in the sequence. The assignment

$${t}_{i , s , c} ← {max}_{{s}^{′} ∈ S} \{ l ( [ \begin{matrix}{x}_{i} \\ o ( s )\end{matrix} ] \vert [ \begin{matrix}{x}_{i - 1} \\ o ( {s}^{′} ) \\ o ( c )\end{matrix} ] , γ : τ , {γ}^{′} : θ , \dot{μ} , \dot{Σ} ) + {t}_{i - 1 , {s}^{′} c} \} sets {t}_{i , s , c}$$

based on a conditional multivariate Gaussian and t_{i-1,s′,c}, which has been previously determined and stored with dynamic programming.

The term dynamic programming as used by embodiments of the subject matter is that quantities precomputed earlier in the sequence can be used to later in the sequence. Dynamic programming is efficient because of this re-use of precomputed data. More generally, dynamic programming can be used to solve an optimization problem by dividing it into simpler subproblems where an optimal solution to the overall problem is based on an optimal solution to the simpler subproblems. In embodiments of the subject matter, the optimization problem is maximization and “simpler” corresponds values that have been precomputed earlier in the sequence.

Once the values for t have been determined for each class for a given sequence, embodiments of the subject matter can then determine a most likely class based on

$${argmax}_{c ∈ C} \{ {max}_{s ∈ S} \{ {t}_{m , s , c} \} + l ( c ) \} ,$$

where l(c) is the natural logarithm of the probability of class c.

Embodiments of the subject matter can execute the following steps to learn a prediction model, which comprises the parameters μ, Σ, \dot{μ}, \dot{Σ} and l(c). The parameter

$$l ( c ) = ln \frac{{\sum }_{j = 1}^{n} ( {c}_{j} = c )}{n} ,$$

which corresponds to the natural logarithm of the frequency of class c in the data. Here, n corresponds to the number of training examples. Adjustments to this frequency can be made to avoid zero probabilities.

In embodiments of the subject matter, the first step in learning the remaining parameters μ, Σ, \dot{μ} , and \dot{Σ} in the prediction model is to randomly initialize the states for each element in each sequence (training example). This is shown in the box below. Here, mⱼ corresponds to the number of elements in the sequence for training example j, and r_{j,i} corresponds to the state associated with element i in training example j.

$$\# randomly initialize states$$

$$1 \leq j \leq n :$$

$$1 \leq j \leq {m}_{j} :$$

$${r}_{j , i} ← random ( S )$$

$$\# update model$$

$$data ← ∅$$

$$data ← ∅$$

$$1 \leq j \leq n :$$

$$data . append ( [ \begin{matrix}{x}_{j , 1} \\ o ( {r}_{j , 1} ) \\ o ( {c}_{j} )\end{matrix} ] )$$

$$1 \leq j \leq {m}_{j} :$$

$$\dot{data} . append ( [ \begin{matrix}{x}_{j , 1} \\ o ( {r}_{j , 1} ) \\ \begin{matrix}{x}_{j , i - 1} \\ o ( {r}_{j , i - 1} ) \\ o ( {c}_{j} )\end{matrix}\end{matrix} ] )$$

$$μ ← data . mean ( )$$

$$Σ ← data . covariance ( )$$

$$\dot{μ} ← \dot{data} . mean ( )$$

$$\dot{Σ} ← \dot{data} . covariance ( )$$

Next, embodiments of the subject matter can execute the update model box above. The box describes two data stores, data and d\dot{a}ta both of which are initially set to empty (i.e. ø). These data stores can correspond to sets, lists, arrays of data, or any other structure capable of storing and retrieving data. Within the outer loop 1≤j≤n, embodiments of the subject matter first handle the edge cases for each training sequence, where x_{j,i} is the i^{th} element of the j^{th} training example, and cⱼ is the class of the j^{th} training example. In embodiments of the subject matter, the inner loop handles the internal cases for each training sequence (mⱼ is the sequence length of the j^{th} training example).

In either case (edge and interior), the append operation adds to the corresponding example to the training data. Subsequently, when all data has been appended, embodiments of the subject matter can determine the mean and covariance matrices of each set of training data. Multiple ways can be used to determine these matrices. Moreover, to prevent singularity in the covariance matrices, a small value can be added along the diagonal of each covariance matrix.

Embodiments of the subject matter can predict the most likely states for every element of every training example and then update the mean and covariance matrix. These steps are shown in the box below. After embodiments of the subject matter execute the update model box, the next few steps are similar to the prediction method in embodiments of the subject matter, except that the class is known during training. After each training example is processed, embodiments of the subject matter can execute the backtrace box, which determines a most likely sequence of states, which can be subsequently used to update the model (the top of the repeat until convergence box) after all training examples are processed. The backtrace box determines states for the next round of processing in the repeat until convergence box.

The backtrace assignments begin with the last index value, m, in the sequence. Specifically, the assignment

$${r}_{j , {m}_{j}} ← {argmax}_{s ∈ S} \{ {t}_{{m}_{j} , s} \}$$

stores the most likely state for position m in the j^{th} sequence.

Subsequently, mⱼ≤i≤2:r_{j,i-1}←g_{i,r}_{_{j,i}} sets the values for the remaining positions from m down to 1. Because the “for” loop runs from m down to 1, the assignment is based on the previously set index value rᵢ. This is another use of dynamic programming in embodiments of the subject matter.

The steps of model updates, prediction, and backtrace can repeat until convergence. Convergence can be defined in several ways. One way is with a fixed number of iterations of the above routine. Another way is until a difference of an aggregation of

$${max}_{s ∈ S} \{ {t}_{j , {m}_{j} , s} \}$$

over all training examples 1≤j≤n between successive iterations is less than a given threshold. Aggregation functions include but are not limited to sum, mean, min, max. A difference can be absolute or relative. Convergence can also be defined as reaching a local maximum in likelihood.

$$\# repeat until convergence$$

$$update model$$

$$1 \leq j \leq n :$$

$$s ∈ S :$$

$${t}_{1 , s} ← l ( [ \begin{matrix}{x}_{i} \\ o ( s )\end{matrix} ] \vert o ( {c}_{j} ) , γ : τ , θ : θ , μ , Σ )$$

$${g}_{1 , s} ← s$$

$$2 \leq i \leq {m}_{j} :$$

$$s ∈ S :$$

$${t}_{i , s} ← {max}_{{s}^{′} ∈ S} \{ l ( [ \begin{matrix}{x}_{i , j} \\ o ( s )\end{matrix} ] \vert [ \begin{matrix}{x}_{j , i - 1} \\ o ( {s}^{′} ) \\ o ( {c}_{j} )\end{matrix} ] , γ : τ , {γ}^{′} : θ , \dot{μ} , \dot{Σ} ) + {t}_{i - 1 , {s}^{′}} \}$$

$${g}_{i , s} ← {max}_{{s}^{′} ∈ S} \{ l ( [ \begin{matrix}{x}_{i , j} \\ o ( s )\end{matrix} ] \vert [ \begin{matrix}{x}_{j , i - 1} \\ o ( {s}^{′} ) \\ o ( {c}_{j} )\end{matrix} ] , γ : τ , {γ}^{′} : θ , \dot{μ} , \dot{Σ} ) + {t}_{i - 1 , {s}^{′}} \}$$

$$backtrace$$

$$\# backtrace$$

$${r}_{j , {m}_{j}} ← {argmax}_{s ∈ S} \{ {t}_{{m}_{j} , s} \}$$

$${m}_{j} \geq i \geq 2 : {r}_{j , i - 1} ← {g}_{i , {r}_{j , i}}$$

The probability of finding a global maximum likelihood associated with the model can increase with multiple random restarts, which can be run in parallel to result in different model. The model with the largest sum of

$${max}_{s ∈ S} \{ {t}_{j , {m}_{j} , s} \}$$

over all training examples can be chosen as the best model. Alternatively, an ensemble of the top k models can be chosen for prediction. Multiple different ensembling methods can be used to combine them during prediction including choosing the most frequently predicted class across all the ensembles or the most frequent class across weighted ensembles, where the weighting itself can be learned.

Note that a mathematically equivalent version of the assignment for t and g can be defined in terms of a product of probabilities rather than a sum of log of the probabilities. The product of probabilities can result in extremely low numbers, which can cause hardware underflow. A preferred embodiment of the subject matter uses the sum of the natural logarithm of the probabilities. Moreover, with this form, the multivariate Gaussian distribution simplifies so that no exponentials are required. Other mathematically equivalent expressions can be used as well as approximations of the multivariate Gaussian distribution.

An appropriate number of states (as in {1 . . . k}) can be determined in multiple different ways. For example, a validation set of sequences can be reserved and used to evaluate the likelihood of the sequences using an aggregation of

$${max}_{s ∈ S} \{ {t}_{j , {m}_{j} , s} \}$$

over a validation set of examples. Aggregation functions include but are not limited to min, mean, max, and sum. The number of states can be explored from 1 . . . k until a maximum in the likelihood is found (the peak method) or until the likelihood does not significantly increase (the elbow method). These methods are similar to those of finding an appropriate number of mixtures for a Gaussian mixture distribution.

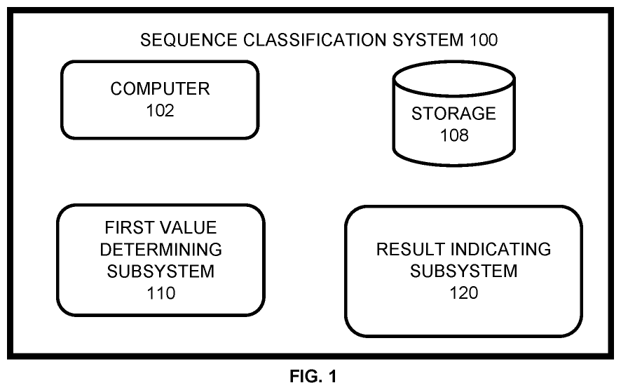

FIG. 1 shows an example sequence classification system 100 in accordance with an embodiment of the subject matter. Sequence classification system 100 is an example of a system implemented as a computer program on one or more computers in one or more locations (shown collectively as computer 102), with one or more storage devices (shown collectively as storage 108), in which the systems, components, and techniques described below can be implemented.

Sequence classification system 100 classifies a sequence of elements where each element comprises one or more continuous values. During operation, sequence classification system 100 determines, with first value determining subsystem 110, a first value indexed by a first state, a first position, and a class, based on data at the first position in the sequence, the first state, the class, a second state, data at a second position in the sequence, and a second value indexed by the second state, the second position, and the class, where the second position is in proximity to the first position, and where the second value indexed by the second state, the second position, and the class was previously determined by dynamic programming.

More specifically, first value determining subsystem 110 determines t_{i,s,c} which corresponds to the first value. The first position corresponds to 1, the first state corresponds to s, and the class corresponds to c. Here, t is indexed by i, s, c. The sequence corresponds to x and data at the first position corresponds to xᵢ. The second state corresponds to s′ and data at the second position corresponds to xᵢ₋₁. The second value indexed by the second state, the second position, and the class corresponds to t_{i-1,s′,c}. Moreover, the second position (i−1) is in proximity to the first position (i) because it differs by only one (1). Also, t_{i-1,s′,c} was previously determined by dynamic programming.

Subsequently, sequence classification system 100 returns a result indicating the class based on the sequence with result indicating subsystem 120. This step corresponds to determining

$${argmax}_{c ∈ C} \{ {max}_{s ∈ S} \{ {t}_{m , s , c} \} + l ( c ) \} ,$$

which returns a most likely class for the sequence, comprising m elements.

The preceding description is presented to enable any person skilled in the art to make and use the subject matter, and is provided in the context of a particular application and its requirements. Various modifications to the disclosed embodiments will be readily apparent to those skilled in the art, and the general principles defined herein may be applied to other embodiments and applications without departing from the spirit and scope of the subject matter. Thus, the subject matter is not limited to the embodiments shown, but is to be accorded the widest scope consistent with the principles and features disclosed herein.

Embodiments of the subject matter and the functional operations described in this specification can be implemented in digital electronic circuitry, in tangibly embodied computer software or firmware, in computer hardware, including the structures disclosed in this specification and their structural equivalents, or in combinations of one or more of them.

Embodiments of the subject matter described in this specification can be implemented as one or more computer programs, i.e., one or more modules of computer program instructions encoded on a tangible non-transitory program carrier for execution by, or to control the operation of data processing system.

A computer program (which may also be referred to or described as a program, software, a software application, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment.

A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code.

Alternatively, or in addition, the program instructions can be encoded on an artificially generated propagated signal, e.g., a machine-generated electrical, optical, or electromagnetic signal, that is generated to encode information for transmission to a suitable receiver system for execution by a data processing system. The computer storage medium can be a machine-readable storage device, a machine-readable storage substrate, a random or serial access memory device, or a combination of one or more of them.

Computers suitable for the execution of a computer program include, by way of example, can be based on general or special purpose microprocessors or both, or any other kind of central processing unit. Generally, a central processing unit will receive instructions and data from a read only memory or a random-access memory or both. The essential elements of a computer are a central processing unit for performing or executing instructions and one or more memory devices for storing instructions and data.

A computer can also be distributed across multiple sites and interconnected by a communication network, executing one or more computer programs to perform functions by operating on input data and generating output.

A computer can also be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Generally, a computer will also include, or be operatively coupled to receive data from or transfer data to, or both, one or more mass storage devices for storing data, e.g., magnetic, magneto optical disks, or optical disks. However, a computer need not have such devices.

The term “data processing system” encompasses all apparatus, devices, and machines for processing data, including by way of example a programmable processor, a computer, or multiple processors or computers.

For a system of one or more computers to be configured to perform particular operations or actions means that the system has installed on it in software, firmware, hardware, or a combination of them that in operation cause the system to perform the operations or actions. For one or more computer programs to be configured to perform particular operations or actions means that the one or more programs include instructions that, when executed by data processing system, cause the system to perform the operations or actions.

The processor and the memory can be supplemented by, or incorporated in, special purpose logic circuitry. More generally, the processes and logic flows can also be performed by and be implemented as special purpose logic circuitry, e.g., an FPGA (field programmable gate array) or an ASIC (application specific integrated circuit), a dedicated or shared processor that executes a particular software module or a piece of code at a particular time, and/or other programmable-logic devices now known or later developed. When the hardware modules or system are activated, they perform the methods and processes included within them.

The system can also include, in addition to hardware, code that creates an execution environment for the computer program in question, e.g., code that constitutes processor firmware, a protocol stack, a database management system, an operating system, or a combination of one or more of them.

The computer-readable storage medium includes, but is not limited to, volatile memory, non-volatile memory, magnetic and optical storage devices such as disk drives, magnetic tape, CDs (compact discs), DVDs (digital versatile discs or digital video discs), computer instruction signals embodied in a transmission medium (with or without a carrier wave upon which the signals are modulated), and other media capable of storing computer-readable media now known or later developed. For example, the transmission medium may include a communications network, such as a LAN, a WAN, or the Internet.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

The methods and processes described in the detailed description section can be embodied as code and/or data, which can be stored in a computer-readable storage medium as described above. When a computer system reads and executes the code and/or data stored on the computer-readable storage medium 120, the computer system performs the methods and processes embodied as data structures and code and stored within the computer-readable storage medium.

The components of the system can be interconnected by any form or medium of digital data communication, e.g., a communication network. Examples of communication networks include a local area network (“LAN”) and a wide area network (“WAN”), e.g., the Internet.

While this specification contains many specific implementation details, these should not be construed as limitations on the scope of any subject matter or of what may be claimed, but rather as descriptions of features that may be specific to particular embodiments of particular subject matters. Certain features that are described in this specification in the context of separate embodiments can also be implemented in combination in a single embodiment.

Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system modules and components in the embodiments described above should not be understood as requiring such separation in all embodiments, and it should be understood that the described program components and systems can generally be integrated together in a single software product or packaged into multiple software products.

The foregoing descriptions of embodiments of the subject matter have been presented only for purposes of illustration and description. They are not intended to be exhaustive or to limit the subject matter to the forms disclosed. Accordingly, many modifications and variations will be apparent to practitioners skilled in the art. Additionally, the above disclosure is not intended to limit the subject matter. The scope of the subject matter is defined by the appended claims.

What is claimed is:

1. A computer-implemented method for facilitating classifying a sequence comprising:
determining a first value indexed by a first state, a first position, and a class, based on data at the first position in the sequence, the first state, the class, a second state, data at a second position in the sequence, and a second value indexed by the second state, the second position, and the class,
wherein the second position is in proximity to the first position, and wherein the second value indexed by the second state, the second position, and the class was previously determined by dynamic programming; and
returning a result indicating the class based on the sequence.

2. The method of claim 1,
wherein determining the first value is based on a multivariate Gaussian distribution comprising a mean vector and a covariance matrix.

3. The method of claim 2,
wherein the mean vector and covariance matrix are learned from training data comprising at least one sequence.

4. The method of claim 3,
wherein the mean vector and the covariance matrix are learned from training data comprising a first one-hot representation of the first state, a second one-hot representation of the second state, and a third one-hot representation of the class.

5. One or more non-transitory computer-readable storage media storing instructions that when executed by one or more computers cause the one or more computers to perform operations for facilitating classifying a sequence, comprising:
determining a first value indexed by a first state, a first position, and a class, based on data at the first position in the sequence, the first state, the class, a second state, data at a second position in the sequence, and a second value indexed by the second state, the second position, and the class,
wherein the second position is in proximity to the first position, and wherein the second value indexed by the second state, the second position, and the class was previously determined by dynamic programming; and
returning a result indicating the class based on the sequence.

6. The one or more non-transitory computer-readable storage media of claim 5,
wherein determining the first value is based on a multivariate Gaussian distribution comprising a mean vector and a covariance matrix.

7. The one or more non-transitory computer-readable storage media of claim 6,
wherein the mean vector and covariance matrix are learned from training data comprising at least one sequence.

8. The one or more non-transitory computer-readable storage media of claim 7,
wherein the mean vector and the covariance matrix are learned from training data comprising a first one-hot representation of the first state, a second one-hot representation of the second state, and a third one-hot representation of the class.

9. A system comprising one or more computers and one or more storage devices storing instructions that when executed by the one or more computers cause the one or more computers to perform operations for facilitating classifying a sequence, comprising:
determining a first value indexed by a first state, a first position, and a class, based on data at the first position in the sequence, the first state, the class, a second state, data at a second position in the sequence, and a second value indexed by the second state, the second position, and the class,
wherein the second position is in proximity to the first position, and wherein the second value indexed by the second state, the second position, and the class was previously determined by dynamic programming; and
returning a result indicating the class based on the sequence.

10. The system of claim 9,
wherein determining the first value is based on a multivariate Gaussian distribution comprising a mean vector and a covariance matrix.

11. The system of claim 10,
wherein the mean vector and covariance matrix are learned from training data comprising at least one sequence.

12. The system of claim 11,
wherein the mean vector and the covariance matrix are learned from training data comprising a first one-hot representation of the first state, a second one-hot representation of the second state, and a third one-hot representation of the class.
